SEQUENCE LISTING (5 sequences)

SEQ ID NO: 1 - protein, 21 aa, Artificial Sequence
  Description of Artificial Sequence; note=synthetic construct
MEVGWYRSPFSRVVHLYRNGK

SEQ ID NO: 2 - protein, 22 aa, Artificial Sequence
  Description of Artificial Sequence; note=synthetic construct
SIAFPSKTSASIGSLCADARMY

SEQ ID NO: 3 - protein, 13 aa, Artificial Sequence
  Description of Artificial Sequence; note=synthetic construct
HCLGKWLGHPDKF

SEQ ID NO: 4 - protein, 21 aa, Artificial Sequence
  Description of Artificial Sequence; note=synthetic construct
ERVCHCLGKWLGHPDKFVGIT

SEQ ID NO: 5 - protein, 11 aa, Artificial Sequence
  Description of Artificial Sequence; note=synthetic construct
ASQKRPSQRSK

(19) United States
(12) Patent Application Publication
(10) Pub. No.: US 2008/0206143 A1
(43) Pub. Date: Aug. 28, 2008

(54) Blockade of Elr+Cxc Chemokines as a Treatment For Inflammatory and Autoimmune Disease

(75) Inventors: Benjamin Segal, Rochester, NY (US); Thaddeus Carlson, Rochester, NY (US)

Correspondence Address: FISH & RICHARDSON P.C., P.O BOX 1022, Minneapolis, MN, 55440-1022, US

(73) Assignee: University of Rochester, Rochester, NY (US)

(21) Appl. No.: 11/813,318

(22) PCT Filed: Jan. 4, 2006
(86) PCT No.: PCT/US2006/000108

§ 371 Date: Nov. 19, 2007

Related U.S. Application Data
(60) Provisional application No. 60/641,323, filed on Jan. 4, 2005.

(51) Int. Cl. A61K 49/00; A61K 39/395; G01N 33/68; A61P 37/00; G01N 33/53

(52) U.S. Cl. 424/9.2; 424/158.1; 436/86; 435/7.2

(57) ABSTRACT

Experimental autoimmune encephalomyelitis (EAE) is a ThI-mediated autoimmune disease of the central nervous system that is widely used as an animal model of multiple sclerosis (MS). Herein it is demonstrated that CXCR2, a chemokine receptor involved in the recruitment of neutrophils, is expressed in tissues with EAE lesions. Blockade or deficiency of CXCR2 reduces the infiltration of neutrophils to sites of inflammation. Thus provided herein are reagents that antagonize or inhibit ELR+CXC chemokines and methods of use of these reagents in preventing and treating organ-specific autoimmune diseases like multiple sclerosis, and methods or treating various inflammatory conditions and diseases.

This application claims the benefit of U.S. Provisional Application No. 60/641,323 filed Jan. 4, 2005, which is incorporated herein by reference in its entirety.

This invention was made with government support under National Institutes of Health Grant Nos. NS 41562-1 and NS 047687-01A from the National Institute of Neurologic Disorders and Stroke. The government has certain rights in the invention

### BACKGROUND OF THE INVENTION

The majority of autoimmune diseases are chronic conditions, characterized by persistent or relapsing inflammation in the target organ. This is true of multiple sclerosis (MS), an inflammatory disease of central nervous system (CNS) white matter, that generally presents with recurrent episodes of neurological dysfunction followed by a secondary stage of gradually worsening disability. Experimental autoimmune encephalomyelitis (EAE), an animal model with strong pathological similarities to MS, also follows a relapsing, progressive clinical course (Raine, C. S., et al. 1984. Laboratory Investigation 51:534-546). Following acute exacerbations inflammation eventually recedes in individual MS and EAE lesions. However, it is not uncommon for chronic lesions to re-inflame at a subsequent time point and new lesions inevitably form in distinct areas within the CNS white matter. Relatively little is known about the pathological mechanisms responsible for the establishment and re-inflammation of CNS demyelinating lesions over the course of the disease process. Needed in the art are agents that affect these mechanisms.

### SUMMARY OF THE INVENTION

In accordance with the purposes of this invention, as embodied and broadly described herein, this invention, in one aspect, relates to methods of treating or preventing an autoimmune disease. Also provided herein are screening methods for the identification of agents that inhibit the interaction of ELR+CXC chemokines with a receptor, or agents that inhibit the production of ELR+CXC chemokines.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows that neutrophils are present in the spinal cord lesions of mice with EAE. The left panel shows a representative section from a naïve mouse without EAE. The right panel shows a representative sample from a C57BL/6 (A) or Balb/c (B) mouse with EAE. At the peak of disease, mice were formalin fixed and their spinal cords removed for histology. Paraffin embedded sections were giemsa stained. Neutrophils were found in spinal cord infiltrates of mice with acute EAE, as indicated by red arrows. Representative sections are shown.

FIG. 2 shows flow cytometric analysis showing that neutrophils are present in the spinal cord lesions of C57BL/6 mice with EAE. Infiltrating immune cells were isolated from spinal cords of naïve mice, or mice with EAE, by density gradient centrifugation. The left panel shows the results from FACS analysis from pooled naïve spinal cords and the right panel shows results from mice with EAE. Cells were gated on MHC class II-cells. Cells fluorescently labeled for both Ly6G and 7/4 are neutrophils.

FIG. 3 shows that CXCR2, KC, and MIP-2 transcripts are upregulated in the spinal cord during EAE. FIG. 3A shows an RNase protection assay performed using spinal cords from representative mice with EAE and asymptomatic controls. FIG. 3B shows the mean expression of MIP-2, KC and CXCR2 in spinal cords from mice with EAE and controls. The bands shown in (A) were measured by densitometry to quantify mRNA expression of the chemokines and their receptor. Each lane represents mRNA from a spinal cord harvested from an individual mouse at peak disease in immunized mice, or from an age matched naïve mouse. Chemokine and chemokine receptor mRNA expression were normalized to the housekeeping gene L32.

FIG. 4 shows CXC2−/− mice are resistant to EAE. Balb/c CXCR2+/− and CXCR2−/− mice were immunized with 400 μg PLP₁₈₅₋₂₀₆ emulsified in CFA (5 mg/ml M. tuberculosis), and received injections of Bordatella pertussis toxin (300 ng i.p.) on days 0 and 2 post immunization. FIGS. 4 A, B and C each represent an individual experiment, which is internally controlled, with 5-10 mice/group. Mice are rated for degree of paralysis on a 5 point scale of disease severity by an examiner who is blinded to group identity. A score of 1 indicates limp tail; 2 indicates mild hind limb paresis with a waddling gait and frequent missteps on a cage top grate; 3 indicates more severe hind limb paresis with obvious dragging of at least 1 limb; 4 indicates hind limb paralysis and 5 is moribund with 20% or greater weight loss.

FIG. 5 shows that neutrophils are present in the spinal cord lesions of Balb/c CXCR2+/−(left panel) but not Balb/c CXCR2−/− (right panel) mice following immunization with PLP₁₈₅₋₂₀₆. Mice were followed for the development of clinical signs of disease. At the peak of disease, CXCR2+/− mice with EAE and their time point-matched CXCR2−/− counterparts were formalin fixed and their spinal cords removed for histology. Paraffin embedded sections were giemsa stained. Neutrophils were found in spinal cord infiltrates of mice with acute EAE, as indicated by arrows.

FIG. 6 shows that markers of inflammation, including ELR+CXC chemokines are expressed early before EAE onset. SJL mice were immunized with PLP₁₃₉₋₁₅₁+CFA and spinal cords were harvested on days 8-12 post immunization. RNA was isolated from the spinal cords and real time RT-PCR was performed to determine expression levels of mRNA. Target genes were normalized to β-actin and expression levels are presented as fold-induction comparing PLP-immunized mice against mRNA levels in naïve spinal cords. Each data point is the mean value of 4-5 spinal cords per group.

FIG. 7 shows that cytokine secretion is similar between WT and CXCR2−/− mice. BALB/c WT or CXCR2−/− mice were immunized with PLP₁₈₅₋₂₀₆+CFA. On Day 12 post immunization lymph nodes and spleens were harvested and CD4+ T cells were purified by negative selection columns. WT naïve T-depleted splenocytes were used as antigen presenting cells. Proliferation was assessed by 3[H]-thymidine incorporation, and frequency of cytokine secreting cells was assessed by ELISPOT assay.

FIG. 8 shows that T cell proliferation and cytokine secretion are similar between WT and CXCR2−/− mice. BALB/c WT or CXCR2−/− mice were immunized with PLP₁₈₅₋₂₀₆+CFA. On Day 12 post immunization lymph nodes and spleens were harvested and CD4+ T cells were purified by negative selection columns. WT naïve T-depleted splenocytes were used as antigen presenting cells. Frequency of cytokine secreting cells was assessed by ELISPOT assay.

FIG. 9 shows that RB6 treatment depletes neutrophils from the peripheral blood and prevents the onset of EAE. The monoclonal antibody RB6 or control IgG was injected i.p. into mice immunized with PLP₁₃₉₋₁₅₁+CFA starting on day 8 post-immunization. (Note: RB6 targets the cell surface marker Gr-1 that is expressed at high levels on neutrophils). Peripheral blood was harvested on day 13 and stained for flow cytometry to confirm depletion of neutrophils (left 2 panels). Cells staining positive for both 7/4 and CD11b are considered neutrophils. Mice were also followed for the development of clinical signs of EAE (right panel).

FIG. 10 shows that T cell proliferation is similar between RB6- and IgG control-treated mice mice. SJL mice were immunized with PLP₁₃₉₋₁₅₁+CFA and injected with control IgG or RB6 starting on day 8 post immunization. On Day 13 post immunization lymph nodes and spleens were harvested and CD4+ T cells were purified by negative selection columns. WT naïve T-depleted splenocytes were used as antigen presenting cells. Proliferation was assessed by 3[H]-thymidine incorporation.

FIG. 11 shows that cytokine secretion is similar between RB6- and IgG control-treated mice mice. SJL mice were immunized with PLP₁₃₉₋₁₅₁+CFA and injected with control IgG or RB6 starting on day 8 post immunization. On Day 13 post immunization lymph nodes and spleens were harvested and CD4+ T cells were purified by negative selection columns. WT naïve T-depleted splenocytes were used as antigen presenting cells. Frequency of cytokine secreting cells was assessed by ELISPOT assay.

### DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Disclosed herein are methods for preventing or treating an organ-specific autoimmune disease in a subject comprising administering to the subject an agent that blocks binding of an ELR+(positive) CXC chemokine with a receptor. The agent can be administered to a subject at risk for an organ-specific autoimmune disease or a subject with an organ-specific autoimmune disease.

The methods disclosed herein can be used for preventing a disease or condition. Herein, “preventing” refers to any reduction in the onset of a disease or condition by reducing the severity or delaying the onset of one or more symptoms. It is understood and herein contemplated that “treating” refers to the reduction or cessation of disease progression. With regard to multiple sclerosis, reduction or cessation of disease progression includes the following: Slowing the rate of progression of clinical disability (ex. As measured by the expanded disability severity scale or the multiple sclerosis multifunctional composite); decreasing the frequency of clinical exacerbations; slowing the rate of tissue destruction and/or lesion formation documented by radiological imaging (ex. the rate of atrophy/white matter tissue loss, accumulation of T2 or FLAIR lesions, and/or frequency of gadolinium enhancing lesions measured by serial MRI scans of the brain and/or spinal cord); accelerating the rate of clinical recovery from an acute exacerbation; accelerating the resolution of a gadolinium enhancing lesion (as measured by serial MRI scanning); blocking or slowing the progression of dementia as assessed by neurophysiological testing. Therefore, in the disclosed methods, “prevention” or “treatment” can refer to a 10%, 20%, 30%, 40%, 50%, 60%, 70%, 80%, 90%, or 100% reduction in the onset or severity of an established disease or the disease progression. For example, the disclosed methods can be used to prevent or treat multiple sclerosis. It is understood and herein contemplated that the prevention or treatment of multiple sclerosis as disclosed herein can mean a 10% delay in the onset of symptoms or reduction in one or more symptoms associated with multiple sclerosis or the complete cessation of recurrent episodes. It is understood and herein contemplated that “prevention” and “treatment” do not necessarily refer to an absence of the establishment of disease or condition or a cure of the disease or condition, but an improvement in the outlook of a disease or condition.

“Chemokines” refers to a family of cytokines with the ability to stimulate and direct the movement of leukocytes. Typically, chemokines are further classified into subfamilies (for example, CC or CXC) based on the pattern of terminal cysteine residues. The methods disclosed herein relate to CXC chemokines. Typically, CXC chemokines are secreted by monocytes, endothelial cells, astrocytes, and fibroblasts and exert their effect on polymorphonuclear leukocytes (PMNLs) such as neutrophils. Effects have also been described on glial cells (i.e., astrocytes) and glial stem cells (such as oligodendroglial progenitor cells). CXC chemokines can be further distinguished by the presence or absence of glutamic acid-lysine-arginine motifs (ELR+ or ELR−, respectively). Typically, ELR+CXC chemokines bind to the CXCR2 or the CXCR1 receptor and act as chemoattractants and activators of PMNLs/neutrophils. Examples of ELR+CXC chemokines include but are not limited to macrophage inflammatory protein-2 (MIP-2), lipopolysaccharide-induced CXC chemokine (LIX), Interleukin-8 (IL-8), KC, neutrophil-activating protein-2 (NAP-2), growth-related oncogenes (GRO)-α, GRO-β, and GRO-γ, granulocyte chemotactic protein-2 (GCP-2), and epithelial neutrophil-activating protein 78 (ENA-78).

ELR+CXC chemokines are potent chemoattractants for polymorphonuclear leukocytes (PMNLs) such as neutrophils. In the context of autoimmune diseases, induction of ELR+CXC chemokines within the target organ would likely result in an influx of PMNLs at disease initiation, relapse and/or progression. PMNLs could, in turn, promote the subsequent recruitment of lymphoid and myeloid cells to inflammatory foci via a variety of mechanisms including: (i) the secretion of chemokines that attract lymphocytes and/or monocytes (such as MIP-1α); (ii) the release of enzymes, such as metalloproteinases, that modify the basement membrane/extracellular matrix in a manner that affords easier penetration of effector leukocytes into the perivascular space/tissue parenchyma; and (iii) the release of factors, such as histamine, that increase vascular permeability.

Disclosed herein are methods for preventing an organ-specific autoimmune disease in a subject at risk for an organ-specific autoimmune disease comprising administering to the subject at risk for an organ-specific autoimmune disease an agent that blocks binding of an ELR+ (positive) CXC chemokine with a receptor, wherein the ELR+CXC chemokine is selected from the group of chemokines consisting of MIP-2, lipopolysaccharide-induced CXC chemokine (LIX), Interleukin-8 (IL-8), KC, neutrophil-activating protein-2 (NAP-2), growth-related oncogenes (GRO)-α, GRO-β, and GRO-γ, granulocyte chemotactic protein-2 (GCP-2), and epithelial neutrophil-activating protein 78 (ENA-78) or any combination thereof. Also disclosed are methods, wherein the receptor is CXCR2, CXCR1, or a combination thereof.

The disclosed methods can be used for the treatment of organ-specific autoimmune diseases and inflammatory conditions. Such diseases are well-known in the art and include but are not limited to rheumatoid arthritis, multiple sclerosis, acute disseminated encephalomyelitis, optic neuritis, transverse myelitis Sjögren's syndrome, Inflammatory Bowel Disease (IBD), diabetes, uveitis, thyroiditis, psoriasis, psoriatic arthritis, myasthenia gravis, paraneoplastic syndromes, Rasmussen's encephalitis, chronic inflammatory demyelinating polyneuropathy, systemic lupus erythematosis, sarcoidosis, Bechet's disease, vasculitides, and Guillain-Barre syndrome. Thus specifically disclosed are methods for preventing or treating an organ-specific autoimmune disease, wherein the autoimmune disease is selected from the group of autoimmune diseases consisting of rheumatoid arthritis, multiple sclerosis, acute disseminated encephalomyelitis, optic neuritis, transverse myelitis, uveitis, Sjögren's syndrome, IBD, diabetes, thyroiditis, psoriasis, psoriatic arthritis, myasthenia gravis, paraneoplastic syndromes, Rasmussen's encephalitis, chronic inflammatory demyelinating polyneuropathy, systemic lupus erythematosis, sarcoidosis, Bechet's disease, vasculitides and Guillain-Barre syndrome.

“Agent” refers to any composition including but not limited to antibodies, siRNA, chemical compositions, cytokines, chemokines, or small molecules. The agents of the invention can be prepared as pharmaceutical compositions and combined with adjuvants to increase their effect. For example, the agent can comprise an antibody that blocks the action of ELR+CXC chemokine i.e. by blocking binding of the chemokine receptor. Thus also disclosed are methods, wherein the agent is a neutralizing antibody to MIP-2. Similarly, the agents may also comprise antibodies to other chemokines or chemokine receptors. Therefore, one embodiment of the disclosed methods are methods, wherein the agent is an antibody to CXCR2 or CXCR1 and wherein the antibody blocks MIP-2, LIX, IL-8, KC, NAP-2, GRO-α, GRO-B, GRO-γ, GCP-2, or ENA-78 binding without causing signaling through CXCR2 and/or CXCR1. Similarly, the agents can include, but are not limited to antibodies that bind ELR+CXC chemokines, such as LIX, IL-8, MIP-2, KC, NAP-2, GRO-α, GRO-B, GRO-γ, GCP-2, and ENA-78. These antibodies include neutralizing antibodies that can prevent LIX, IL-8, M1P-2, KC, NAP-2, GRO-α, GRO-B, GRO-γ, GCP-2, and ENA-78 from binding to its ligand CXCR2 and/or CXCR1 (i.e., blocking antibody). It is understood that the antibody can be a polyclonal or monoclonal antibody or antigenic fragments thereof. The antibody can also be a single chain variable region, dimeric antibody, or trimeric antibody. The antibody or antibody fragment can be used as a fusion protein. It is understood that the disclosed agents can comprise both membrane bound and soluble forms of chemokines, cytokines, ligands, and their receptors or derivatives thereof. Thus, for example, specifically contemplated are methods, wherein the agent is a soluble form of CXCR1 or CXCR2 or a derivative or analog thereof.

The term “subject” is used throughout this disclosure to refer to any organism, tissue, or cell being contacted with the agent or treated with the agent. Such subjects include but are not limited to tissue culture cells, mammals, mice, rats, guinea pigs, dogs, pigs, rabbits, sheep, monkeys, chimpanzees, and humans. It is understood and herein contemplated that the disclosed methods of prevention, inhibition, treating, screening, and diagnosing include methods of prevention, inhibition, treating, screening, and diagnosing, wherein the subject is a mammal.

Herein, “blocks” or “blocking” refers to the interruption of an interaction. It is understood that such interactions may be “blocked” through the action of a competing receptor or ligand. Alternatively, the blocking may occur through steric hindrance. It is also understood and herein contemplated that blocking may occur through the action of an agent that induces a conformational change in a receptor or ligand such that the interaction can not take place. Thus, for example, the interaction of MIP-2 and CXCR2 is considered blocked if an agent with greater affinity for CXCR2 binds to CXCR2 thus preventing the interaction of MIP-2. Another example of blocking is the action of a neutralizing antibody on its ligand. “Blocking” can refer to a complete blockade or a partial blockade of an interaction.

The methods described herein can be used to reduce the exacerbation of an inflammatory condition in a subject. Agents used in the methods inhibit the interaction of LIX, IL-8, MIP-2, KC, NAP-2, GRO-α, GRO-B, GRO-γ, GCP-2, or ENA-78 with CXCR1 or CXCR2. It is understood that the inhibition of the interaction of LIX, IL-8, MIP-2, KC, NAP-2, GRO-α, GRO-B, GRO-γ, GCP-2, or ENA-78 with CXCR1 or CXCR2 can reduce the exacerbation of a disease of condition. The interaction between LIX, IL-8, MIP-2, KC, NAP-2, GRO-α, GRO-B, GRO-γ, GCP-2, and ENA-78 with CXCR1 or CXCR2 provides the signaling through which neutrophils are drawn to an area of inflammation or an organ-specific autoimmune disease. Thus any agent that blocks this interaction can be used in the present methods. For example, specifically disclosed and herein contemplate are methods of treating a subject with an organ-specific autoimmune disease or inflammatory condition comprising administering to the subject an effective amount of an agent that inhibits the interaction of MIP-2 or CXCR2, wherein the agent is an antibody to MIP-2 and wherein the antibody blocks MIP-2 binding without causing signaling through CXCR2. Optionally, the agent comprises a small organic molecule or a macromolecule that binds to either MIP-2 or CXCR2 so as to inhibit their interaction.

Herein “inhibition,” “inhibits,” or “inhibiting” refer to the modulation of a cell, interaction, or action in a reducing manner. It is understood that “inhibition” can refer to any decrease in an action or activity of a cell, or as cellular interaction, or molecular interaction, or action including but not limited to the complete ablation of the action, interaction, or activity. For example, inhibition of an autoimmune disease includes delaying the onset or decreasing the severity of at least one symptom of the autoimmune disease by 5%, 10%, 20%, 30%, 40%, 40%, 50%, 60%, 70%, 80%, 90%, or 100% or any point in between. Thus, for example, an agent that inhibits an autoimmune disease refers to any method that can decrease the severity of the autoimmune disease by as little as 5% of the severity of the autoimmune disease as well as methods that completely ablate the autoimmune disease.

Disclosed herein are methods of inhibiting an inflammatory condition comprising administering to a subject an agent that blocks the interaction of an ELR+(positive) CXC chemokine with its receptor, and wherein the inflammatory condition is selected from the group of inflammatory conditions consisting of reactive arthritis, spondylarthritis, systemic vasculitis, insulin dependent diabetes mellitus, graft versus host disease, inflammatory bowel disease including Crohn's disease, ulcerative colitis, ischemia reperfusion injury, myocardial infarction, Alzheimer's disease, transplant rejection (allogeneic and xenogeneic), thermal trauma, any immune complex-induced inflammation, glomerulonephritis, myasthenia gravis, anaphylaxis, catheter reactions, atheroma, infertility, thyroiditis, ARDS, post-bypass syndrome, hemodialysis, juvenile rheumatoid arthritis, psoriasis, psoriatic arthritis, systemic lupus erythematosis, Behcets syndrome, hemolytic anemia, pemphigus, bullous pemphigoid, stroke, atherosclerosis, sarcoidoses and scleroderma.

The infiltration of neutrophils/polymorphonuclear leukocytes results in many of the symptoms associated with an autoimmune disease or inflammatory condition. For example, an influx of neutrophils is associated with increased vascular permeability, recruitment of lymphocytes and myeloid cells across the blood-brain-barrier and demyelination of neuronal tissue. Thus, preventing the infiltration of neutrophils to a tissue can be used to treat a subject with an autoimmune disease or inflammatory condition or to prevent the disease or condition. It is also understood that particular interactions or chemokines are discussed that are downstream of an earlier interaction. Specifically disclosed and herein contemplated are methods of inhibiting the production of an ELR+CXC chemokine or the interaction of an ELR+CXC chemokine with a receptor. Also disclosed are methods of preventing the infiltration of neutrophils into a tissue comprising inhibiting the interaction of ELR+CXC chemokines with a receptor by inhibiting production of the ELR+CXC chemokine, wherein the production of the ELR+CXC chemokine is inhibited by blocking IL-23 from binding IL-23R. Also disclosed are methods of inhibition, wherein IL-23 is blocked by binding IL-23 with an anti-IL-23 antibody.

Disclosed are methods of preventing the infiltration of neutrophils into a tissue comprising inhibiting the interaction of ELR+CXC chemokines with a receptor by inhibiting production of the ELR+CXC chemokine, wherein the production of the ELR+CXC chemokine is inhibited by blocking IL-17 from binding IL-17R. Also disclosed are methods of inhibition, wherein IL-17 is blocked by binding IL-17 with an anti-IL-17 antibody.

It is well known in the art that some autoimmune diseases and inflammatory conditions are chronic in nature. Moreover, it is understood that some chronic autoimmune diseases and inflammatory conditions can appear to be under control, but re-emerge or relapse. The methods taught herein can be used at various points prior to and during the course of the disease or condition. Thus specifically contemplated are methods of treating or preventing an autoimmune diseases and inflammatory response or condition in a subject, comprising administering to the subject in need thereof an effective amount of an agent that inhibits ELR+CXC chemokines, wherein the agent is administered after the inflammatory response or condition has been initially induced but before a first relapse. Also disclosed are methods of treatment or prevention of an autoimmune diseases and inflammatory response or condition, wherein the agent is administered at the time of a first relapse. Also disclosed are methods of treatment or prevention, wherein the agent administered prevents the progression of the chronic inflammatory condition or autoimmune disease. Also disclosed are methods of treatment or prevention of an inflammatory response or condition, wherein the agent is administered at the time of, or following, the initial clinical exacerbation (i.e., the presenting episode) but prior to a first clinical relapse. Also disclosed are methods of treatment or prevention of an autoimmune diseases and inflammatory response or condition, wherein the agent is administered when subclinical inflammatory activity has been detected (e.g. inflammatory activity as detected by only MRI scans or blood tests) that is likely to evolve into a clinical syndrome in the future. Also disclosed are methods of treatment or prevention of an autoimmune diseases and inflammatory response or condition, wherein the agent is administered after a first relapse. For example, specifically contemplated are methods of treatment or prevention of the invention, wherein said administration is performed at the time of relapse of a chronic neuroinflammatory condition. Also disclosed are method of inhibiting the binding or other interactions between LIX, IL-8, MIP-2, KC, NAP-2, GRO-α, GRO-B, GRO-γ, GCP-2, or ENA-78 and a CXCR1 or CXCR2-expressing cell that can participate in the induction, progression or expression of a autoimmune disease, comprising providing to said cell an amount of an agent effective in inhibiting LIX, IL-8, MIP-2, KC, NAP-2, GRO-α, GRO-B, GRO-γ, GCP-2, or ENA-78 binding to said cell or receptor.

It is well-known and understood that administration of an agent to treat an inflammatory condition may not be curative but may reduce the inflammation and thus may be needed for the life of the subject or until the inflammatory condition is eliminated. Thus also disclosed are methods, wherein the agent is administered chronically. Also disclosed are methods of the invention, wherein the administration of said agent aborts the relapse, or results in more complete or more rapid recovery from a first or subsequent relapse. It is also understood and herein contemplated that administration of the disclosed agents can halt the progression of a chronic inflammatory condition. It is also understood that such treatment can prevent further episodes of an inflammatory condition. Thus also disclosed are methods wherein the administration of said agent stabilizes the clinical status of a patient with a chronic inflammatory condition (prevents or reduces feature accumulation of deficits). Such long term administrations are well-known in the art and can involve daily, weekly, or monthly administrations of the agent or alternatively the agent can be administered in a controlled-release or depot formulation.

It is understood that inflammatory conditions can have multiple effects on a subject which result in undesirable symptoms. It is also understood that there are circumstances in which multiple agents will be preferred to single agent administration for the control of inflammatory conditions. Thus specifically disclosed are methods of treating an inflammatory condition wherein the agents of the treatment methods disclosed herein may be administered in combination with one or more additional drugs that are useful for (a) inhibiting the inflammatory response or condition, and/or (b) treating any other undesired symptom. It is recognized that one of skill in the art will be able to determine if combination therapy is preferred over single agent use.

Disclosed herein are methods of screening for agents for treating an autoimmune disease or inflammatory condition comprising contacting a CXC receptor positive cell with the agent to be screened and detecting binding of ELR+CXC chemokines with the CXC receptor, wherein an agent that inhibits the interaction of the chemokine and the receptor can be used to treat the autoimmune disease or inflammatory condition. Agents identified by the screening methods can be used for the methods disclosed herein. It is understood and herein contemplated that numerous methods may be used to detect the binding of a chemokine to a receptor. For example, the detection of binding can be determined by assaying the presence of down-stream molecules or events. Alternatively, binding can be assessed by determining if the agent reduced the severity of the autoimmune disease or inflammatory condition. Binding can also be detected directly by assaying coupling between an agent and a receptor.

Agents identified via the screening methods disclosed herein can be used for the treatment of T cell mediated inflammation specifically providing a treatment for conditions such as multiple sclerosis. Thus, one embodiment of the disclosed invention is a method of treating a subject with multiple sclerosis, comprising administering to the subject a therapeutic amount of the agent identified by the disclosed screening methods. For example, disclosed are methods of treating a subject with MS, comprising administering to the subject a therapeutic amount of the agent identified by the disclosed screening methods.

Reduction in the inflammatory condition or autoimmune disease can be determined by assessing a variety of clinical and laboratory parameters. Such parameters include the frequency and/or size of gadolinium-enhancing lesions detected by brain or spinal cord MRI scans, white matter lesion burden determined by MRI scanning, rate of white matter tissue loss/atrophy determined by MRI scanning, Axonal damage/loss determined by MR spectroscopy, cerebrospinal fluid pleocytosis, cerebrospinal fluid IgG synthesis rate and/or IgG index, cerebrospinal fluid oligoclonal banding, serum anti-myelin antibody titers, serum autoreactive antibody titers, the frequency of neutrophils, C-reactive protein, erythrocyte sedimentation rate and serum biomarkers or surrogate markers.

Agents that can be used in the disclosed treatment, prevention, or inhibition methods can also affect ELR+CXC chemokines by inhibiting production of ELR+CXC chemokines. Thus, disclosed are methods of screening for agents for treating an autoimmune disease comprising administering to a subject with an autoimmune disease the agent and monitoring the level of ELR+CXC chemokines in the affected tissue, wherein a decrease in the level of ELR+CXC chemokines indicates an agent that is effective in treating the autoimmune disease. Alternatively, a cell that secretes ELR+CXC chemokines could be contacted with the agent and the level of secreted chemokine or the level of chemokine mRNA detected.

It is understood and herein contemplated that the disclosed screening methods can be used for numerous autoimmune or inflammatory conditions. Therefore, disclosed herein are methods of screening, wherein the autoimmune disease is selected from the group of autoimmune diseases consisting of rheumatoid arthritis, multiple sclerosis, acute disseminated encephalomyelitis, optic neuritis, transverse myelitis, uveitis, Sjögren's syndrome, IBD, systemic lupus erythematosis, paraneoplastic syndromes, Rasmussen's encephalitis, diabetes, thyroiditis, psoriasis, psoriatic arthritis, chronic inflammatory demyelinating polyneuropathy, systemic lupus erythematosis, sarcoidosis, Bechet's disease, vasculitides, and Guillain-Barre syndrome.

Also disclosed are methods of screening, wherein the tissue is selected from the group consisting of neural tissue (e.g., brain tissue or spinal cord tissue), lymphatic tissue, hepatic tissue, splenic tissue, pulmonary tissue, cardiac tissue, gastric tissue, intestinal tissue, pancreatic tissue, tissue from the thyroid gland, salivary glands, joints, and the skin.

It is understood that the disclosed screening methods can be used in experimental settings. Such settings can require the induction of the inflammatory response or organ-specific autoimmune disease in order for an agent to have inflammation available for inhibition. It is understood that the necessity of inducing the inflammatory response is known to those of skill in the art. That is, those of skill in the art will recognize if the inflammatory response being inhibited needs to be induced and how the induction can occur. Thus, specifically contemplated are methods of screening for an agent that inhibits an inflammatory response in a subject, comprising the steps of a) administering the agent to the subject, b) inducing the inflammatory response in the subject, and c) detecting ELR+CXC chemokines in the subject, wherein a reduction in the level of ELR+CXC chemokines in the subject as compared to a control level indicates an agent that inhibits an inflammatory response or organ-specific autoimmune disease. Optionally step (a) can precede, follow, or occur simultaneously with step (b). Levels of ELR+CXC chemokines can be detected by numerous parameters including but not limited ELISA, ELISPOT, and Flow cytometry (including, for example, intracellular staining or cytokine secretion assays).

Many different inducers are known in the art and one of skill in the art will understand the appropriate inducer to use for the inflammatory response being studied. It is understood that the inflammatory response can be induced by a peptide, polypeptide, or protein. For example, the inducer can be a myelin protein such as myelin basic protein. In the EAE model of MS the inflammatory condition can be induced by proteolipid protein (PLP), myelin oligodendrocyte protein (MOG), myelin basic protein (MBP) or an antigenic fragment thereof (e.g., PLP₁₃₅₋₁₅₅ (SEQ ID NO: 4), PLP₁₃₉₋₁₅₁ (SEQ ID NO: 3), PLP₁₈₅₋₂₀₆ (SEQ ID NO: 2), MBP_{Acl-11} (SEQ ID NO: 5), or MOG₃₅₋₅₅ (SEQ ID NO: 1)).

The disclosed screening methods use LIX, IL-8, MIP-2, KC, NAP-2, GRO-α, GRO-B, GRO-γ, GCP-2, and ENA-78, CXCR1 or CXCR2 as markers to assess inhibition. The art is replete with examples of methods of detecting cellular markers. For example surface markers and their ligands can be detected using antibodies specific to the marker of interest. Therefore specifically disclosed methods of screening for an agent that inhibits an inflammatory response in a subject with an inflammatory response comprising administering to the subject the agent, inducing the inflammatory response when necessary, and detecting the level of LIX, IL-8, MIP-2, KC, NAP-2, GRO-α, GRO-B, GRO-γ, GCP-2, and ENA-78, CXCR1 or CXC1 or CXCR2 in the subject, wherein LIX, IL-8, MIP-2, KC, NAP-2, GRO-α, GRO-B, GRO-γ, GCP-2, and ENA-78, CXCR1 or CXCR2 or CXCR2 is detected by staining the tissue sample with LIX, IL-8, MIP-2, KC, NAP-2, GRO-α, GRO-B, GRO-γ, GCP-2, and ENA-78, CXCR1 or CXCR2 or CXCR2 antibodies respectively, wherein the antibodies are linked directly or indirectly (Thru a secondary or tertiary antibody, for example) to a detectable moiety. Assays used to detect antibodies are well-known in the art and include but are not limited to flow cytometry, immunohistochemistry, ELISA, and ELISPOT.

It is understood that in addition to screening for agents that can be used to treat, prevent, or inhibit an organ-specific autoimmune disease, and the use of said agents to treat, prevent, or inhibit an organ-specific autoimmune disease in a subject, the information disclosed herein can also be used to provide methods of diagnosing an organ-specific autoimmune disease or of detecting the progression of the disease.

Herein, “diagnosing” refers to a method (including differential diagnosis) of identifying the causation of a set of symptoms. Thus, specifically disclosed are methods of diagnosing multiple sclerosis in a subject comprising detecting in the subject's cerebrospinal fluid an increase in the amount of ELR+CXC chemokines as compared to a control. It is understood and herein contemplated that “increase” refers to any measurable change in the amount of a molecule, wherein the change results in a greater number of molecules. Thus, for example, a change in the amount of ELR+CXC chemokines in the cerebrospinal fluid from 5 ng/ml to 50 ng/ml indicates an increase in the level of ELR+CXC chemokines. It is understood that the diagnosing methods disclosed herein can be used to identify the presence of a disease or condition. Alternatively, the diagnosing methods disclosed herein can be used to identify diseases or conditions where the interaction of ELR+CXC chemokines with a receptor leads to a disease or disease progression.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as described herein. The antibodies are tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855, 1984).

Monoclonal antibodies of the invention can be prepared using hybridoma methods, such as those described by Kohler and Milstein, Nature, 256:495, 1975. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods, such as those described in U.S. Pat. No. 4,816,567 (Cabilly et al.). DNA encoding the monoclonal antibodies of the invention can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr Opin Biotechnol 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

The human antibodies of the invention can be prepared using any technique. Examples of techniques for human monoclonal antibody production include those described by Cole et al. (Monoclonal Antibodies and Cancer Therapy, Alan R., Ed. Liss, p. 77, 1985) and by Boerner et al. (J Immunol, 147(1):86-95, 1991). Human antibodies of the invention (and fragments thereof) can also be produced using phage display libraries (Hoogenboom et al., J Mol Biol, 227:381, 1991; Marks et al., J Mol Biol, 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255, 1993; Jakobovits et al., Nature, 362:255-258, 1993; Bruggermann et al., Year in Immunol. 7:33, 1993). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fc, Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525, 1986, Reichmann et al., Nature, 332:323-327, 1988, and Presta, Curr Opin Struct Biol, 2:593-596, 1992).

Methods for humanizing non-human antibodies are well known in the art. For example, humanized antibodies can be generated according to the methods of Winter and co-workers (Jones et al., Nature, 321:522-525, 1986, Riechmann et al., Nature, 332:323-327, 1988, Verhoeyen et al., Science, 239:1534-1536, 1988), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Antibodies of the invention are preferably administered to a subject in a pharmaceutically acceptable carrier. Suitable carriers and their formulations are described in Remington: The Science and Practice of Pharmacy (19th ed.) A. R. Gennaro, Ed., Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped particles, e.g., films, liposomes or microparticles. It will be apparent to those persons skilled in the art that certain carriers may be more preferable depending upon, for instance, the route of administration and concentration of antibody being administered.

The antibodies can be administered to the subject, organ, tissue, or cell by a variety of methods. For example, the antibody can be added to in vitro culture. The antibody can also be administered to a subject, organ, tissue, or cell in situ by injection (e.g., intravenous, intraperitoneal, subcutaneous, intramuscular), or by other methods such as infusion that ensure its delivery to the target in an effective form. Local or intravenous injection is preferred.

Effective dosages and schedules for administering the antibodies may be determined empirically, and making such determinations is within the skill in the art. Those skilled in the art will understand that the dosage of antibodies that must be administered will vary depending on, for example, the subject that will receive the antibody, the route of administration, the particular type of antibody used and other drugs being administered. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., 1985 ch. 22 and pp. 303-357; Smith et al., Antibodies in Human Diagnosis and Therapy, Haber et al., eds., Raven Press, New York, 1977 pp. 365-389. A typical daily dosage of the antibody used alone might range from about 1 μg/kg to up to 100 mg/kg of body weight or more per day, depending on the factors mentioned above.

As described above, the compositions can also be administered in vivo in a pharmaceutically acceptable carrier. By “pharmaceutically acceptable” is meant a material that is not biologically or otherwise undesirable, i.e., the material may be administered to a subject, along with the cell, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), intramuscularly, by intraperitoneally, transdermally, extracorporeally, intranasally, intraarticularly, topically or the like. The exact amount of the compositions required will vary from subject to subject, depending on the species, age, weight and general condition of the subject, the severity of the disorder being treated, the particular cell used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem, 2:447-451, 1991; Bagshawe, K. D., Br J Cancer, 60:275-281, 1989; Bagshawe, et al., Br J Cancer, 58:700-703, 1988) Senter, et al., Bioconjugate Chem, 4:3-9, 1993; Battelli, et al., Cancer Immunol Immunother, 35:421-425, 1992; Pietersz and McKenzie, Immunolog. Reviews, 129:57-80, 1992; and Roffler, et al., Biochem Pharmacol, 42:2062-2065, 1991). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Res., 49:6214-6220, 1989; and Litzinger and Huang, Biochimica et Biophysica Acta, 1104:179-187, 1992). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis have been reviewed (Brown and Greene, DNA Cell Biol 10:6, 399-409, 1991).

The compositions, including antibodies, can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies or agents can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids, and powders. Conventional pharmaceutical carriers, aqueous, powder or oily bases, thickeners, and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Mice.

BALB/c and C57BL/6 mice were obtained from Jackson Laboratories (Bar Harbor, Me.) and NCI Frederick (Fredrick, Md.). CXCR2 deficient mice on the BALB/c background were also obtained from Jackson Laboratories (Bar Harbor, Me.) and bred in the vivarium at the University of Rochester. All animals were housed under specific-pathogen free, barrier facility conditions.

### Induction of EAE by Active Immunization.

C57BL/6 mice were immunized with 100 μg of MOG₃₅₋₅₅ (SEQ ID NO: 1) emulsified in CFA (with 5 mg/ml heat-killed Mycobacteria tuberculosis H37Ra; vol:vol) by subcutaneous (s.c.) injection at four sites over the flanks. Balb/C wildtype, CXC+/− and CXCR2−/− mice were immunized with 400 μg of PLP₁₅₅₋₂₀₆ (SEQ ID NO: 2) emulsified in CFA. Bordetella pertussis toxin (List Laboratories, San Jose, Calif.) was injected intraperitoneally (i.p., 300 ng/mouse) on days 0 and 2 post-challenge. Animals were examined daily for signs of EAE and rated for severity of neurological impairment on a 5 point scale as previously described (Segal, B. M., and Shevach, E. M. 1996. J Exp Med 184:771-775).

### RNA Analysis.

Total RNA was extracted from spinal cords using Trizol reagent (GIBCO BRL). Multiprobe RNase protection assays (RPA) were performed with Riboquant In Vitro Transcription and customized RPA Kits (Pharmingen). RPA products were resolved on a denaturing polyacrylamide gel and quantified by Phosphorimaging.

### Flow Cytometric Analysis.

Spinal cord mononuclear cells (MNCs) were incubated with FCBLOCK (Becton-Dickinson, San Diego, Calif.) and stained with various combinations of fluorochrome-labeled antibodies to mouse Ly6G and 7/4 or with isotype-matched controls (Pharmingen, San Diego, Calif.). Cells were washed twice and fixed with 1% paraformaldehyde in PBS prior to analysis on a Becton-Dickinson FACSCALIBUR instrument with CELLQUEST software.

### Histopathological Studies.

Spinal cords were dissected from mice following intracardiac perfusion with 4% paraformaldehyde. They were then decalcified in IMMUNOCAL (Decal Chemical Corporation, Talhman, N.Y.) and fixed in 10% buffered formalin. Paraffin-embedded sections of the cervical, thoracic and lumbar regions were stained with Giemsa stain for light microscopy.

### Results:

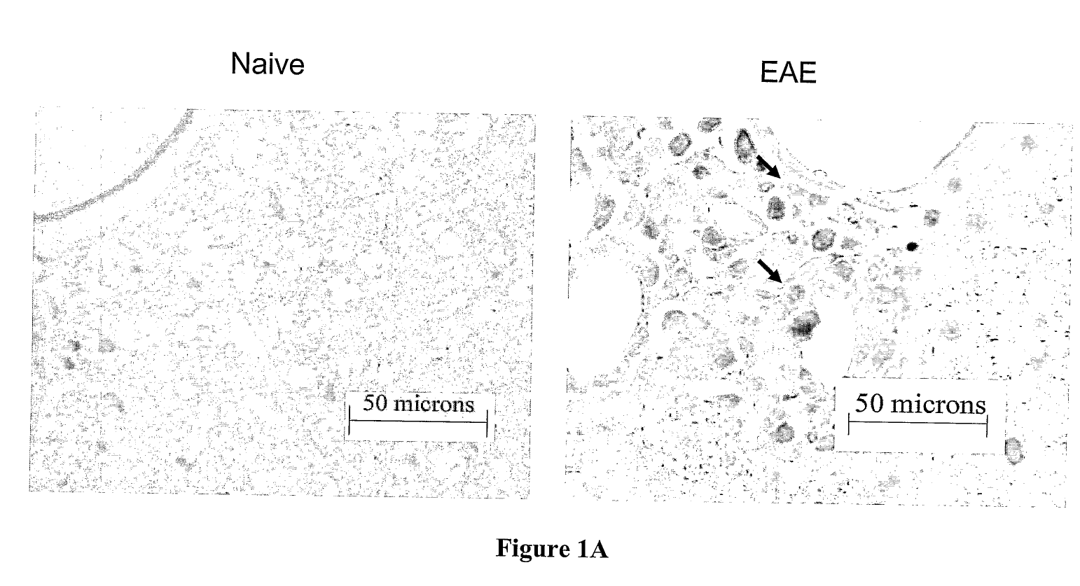

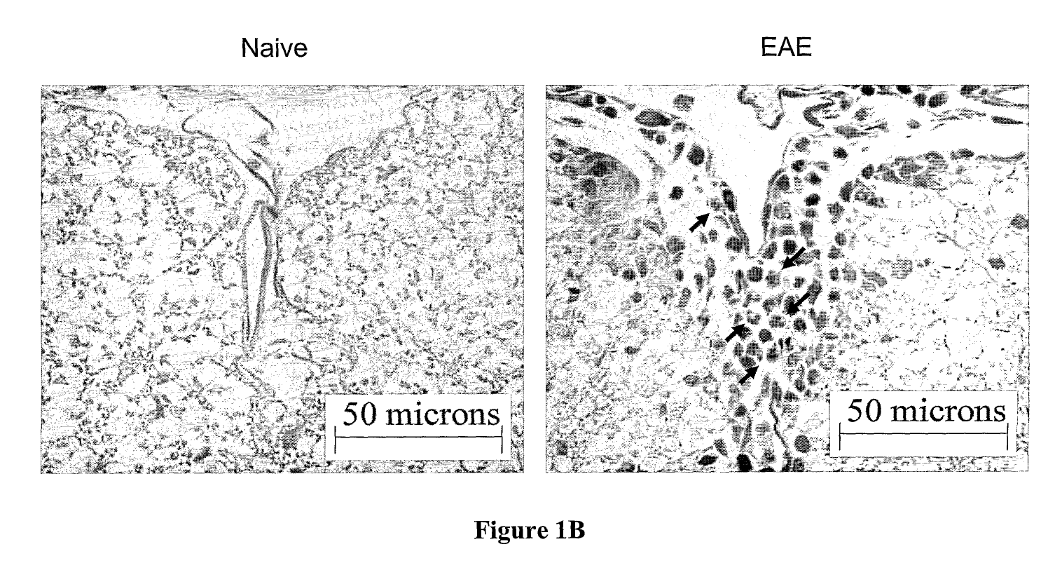

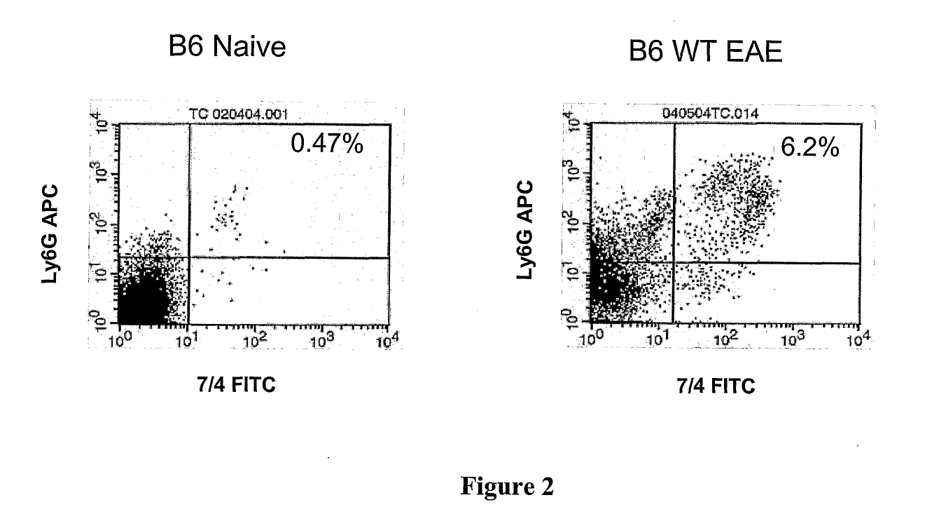

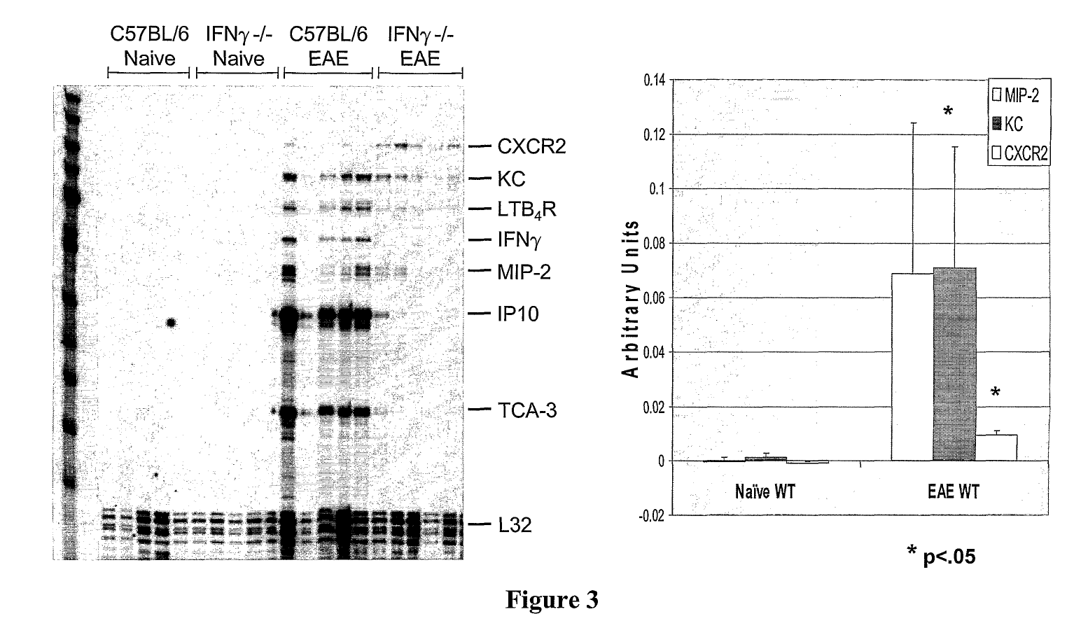

The hypothesis that ELR⁺CXC chemokines are expressed in the CNS during the preclinical phase of EAE resulting in the influx of CXCR2⁺ polymorphonuclear leukocytes across the blood-brain-barrier prior to the onset of clinical deficits was tested. PMNLs could release of factors that increase vascular permeability and attract large numbers of lymphocytes and monocytes to CNS white matter was hypothesized to be important. The infiltration of lymphoid and myeloid cells in such a “secondary wave” leads to demyelination, axonal transaction and, ultimately, neurological deficits. To test this hypothesis, spinal cord infiltrates were analyzed in MOG-sensitized C57BL/6 mice and PLP-sensitized Balb/c mice on the day prior to expected disease onset as well as at peak EAE. Neutrophils were readily detected in spinal cords or sensitized mice at both time points by histopathological and flow cytometric studies (FIGS. 1 and 2). Neutrophils were not detected in spinal cords from untreated control mice or mice immunized with an irrelevant foreign antigent (FIGS. 1, 2). The expression of a panel of ELR+CXC chemokines and their receptor, CXCR2, by RNase protection assays were measured. Transcripts encoding the chemokines KC and MIP-2 were induced in the CNS of sensitized mice during the preclinical phase of EAE and their levels rose at disease onset (FIG. 3). CXCR2 mRNA appeared in conjunction with the CXC chemokines.

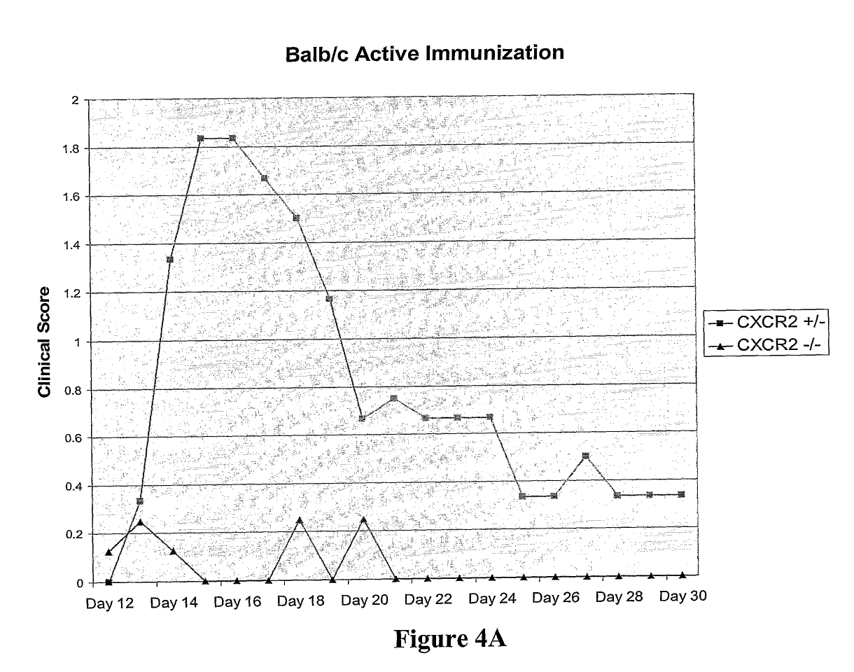

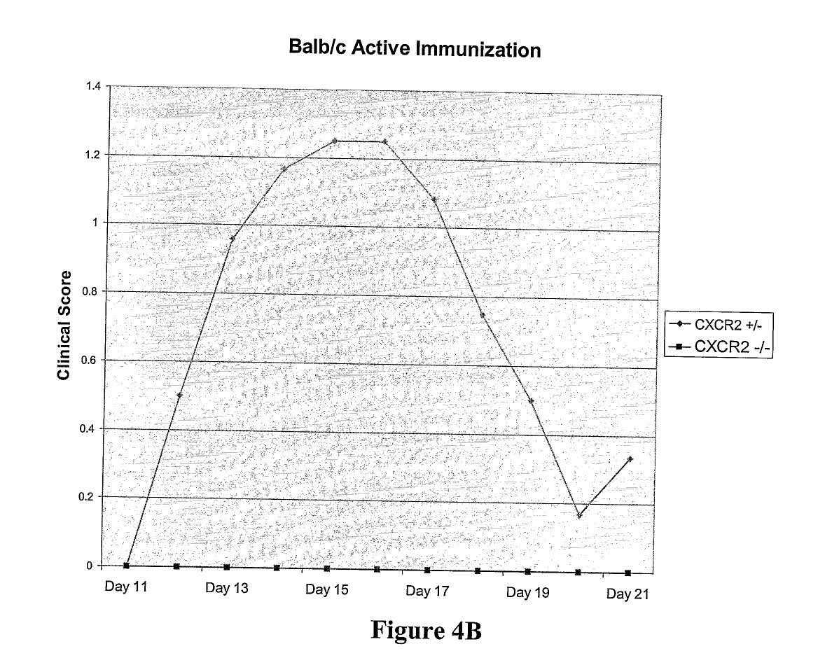

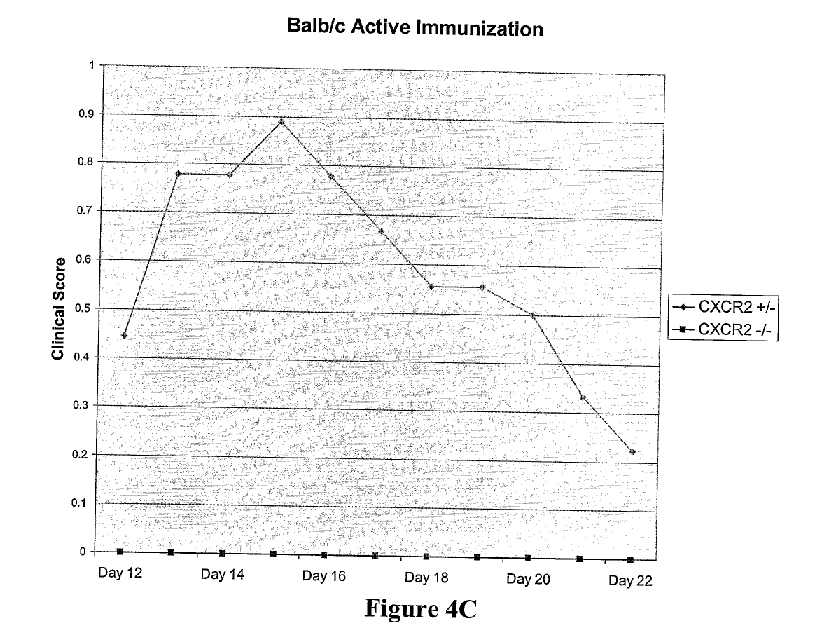

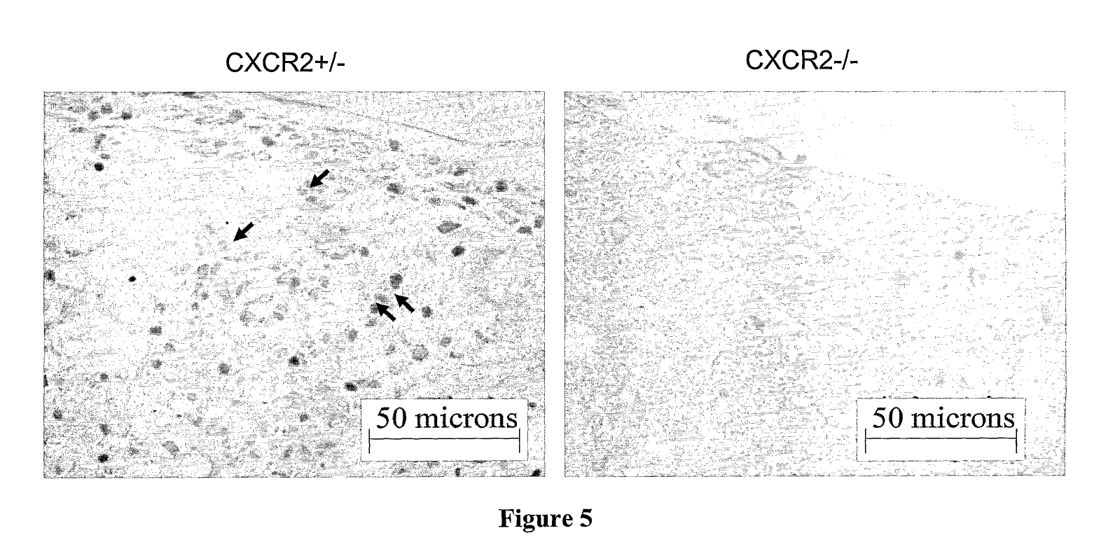

To further characterize the association of CXCR2 with the onset of EAE, CXCR2 deficient (CXCR2−/−) mice were generated and compared with littermate controls (CXCR2+/−) for the manifestation of EAE. Balb/c CXCR2+/− and CXCR2−/− were sensitized with PLP₁₈₅₋₂₀₆ and followed for the development of clinical symptoms of disease in three experiments (FIG. 4 and Table 1). The results indicate that mice deficient in CXCR2 were resistant to EAE. These findings were further underscored via histological evaluation of formalin fixed spinal cord tissue samples (FIG. 5). Giemsa staining shows that neutrophils were present in mice with CXCR2, but not found in CXCR2−/− mice.

TABLE 1
CXCR2−/− mice are resistant to EAE.

| | | | | Mean Peak |
|---|---|---|---|---|
| | | Mean Day of | Mean Peak | Score of Sick |
| | Incidence | Onset | Clinical Score | Mice |
| CXCR2+/− | 13/27 | 13.5 | 1.29 | 2.69 |
| CXCR2−/− | 0/24 | N/A | N/A | N/A |

Balb/c CXCR2+/− and CXCR2−/− mice were immunized with 400 μg PLP₁₈₅₋₂₀₆ emulsified in CFA (5 mg/ml M. tuberculosis), and received injections of Bordatella pertussis toxin (300 ng i.p.) on days 0 and 2 post immunization. Mice were followed for the development of clinical signs of disease and rated on a five point scale of disease severity. These are pooled data from three independent experiments.

Collectively the above data showed that induction of ELR+CXC chemokines in the CNS of neuroantigen-sensitized mice during the preclinical phase of EAE recruits PMNLs to the target organ by CXCR2-dependent pathway. PMNLs accumulated prior to the development of neurological deficits. Furthermore, blockade of CXCR2⁺ leukocyte migration to the CNS was protective against the clinical manifestation of autoimmune encephalomyelitis.

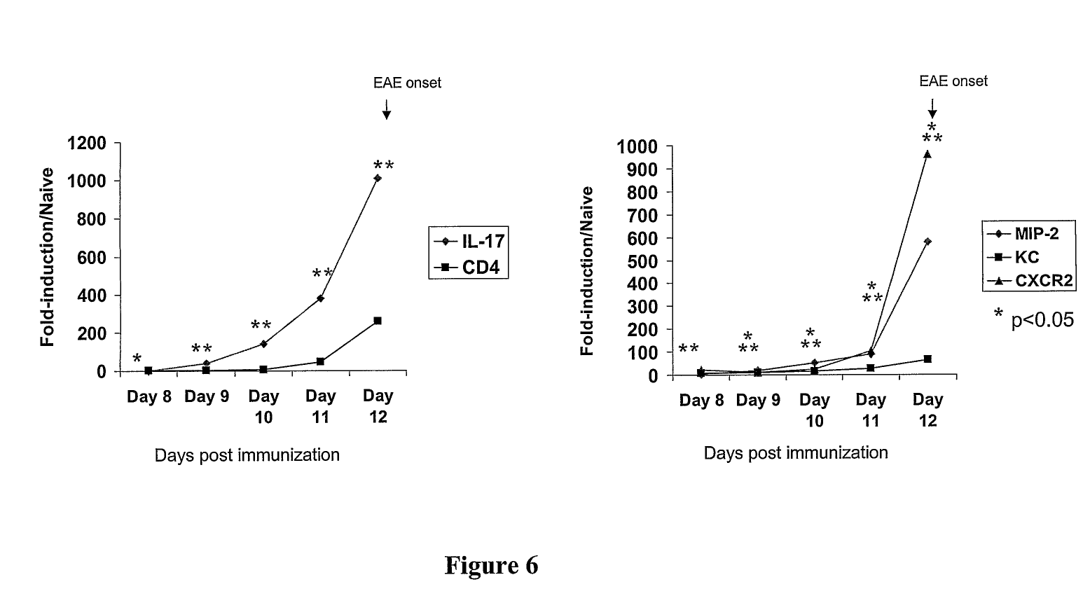

The presence of ELR+CXC chemokines was further characterized by immunizing SJL mice with PLP₁₃₉₋₁₅₁ (SEQ ID NO: 3) and measuring the expression levels of the ELR+CXC chemokines MIP-2, KC, and CXCR2 as well as IL-17 and CD4 relative to naïve controls (FIG. 6). The results show that prior to EAE onset ELR+CXC chemokines expression increased as did the expression of IL-17 and CD4. This indicates that CXCR2⁺ PMNLs have infiltrated the CNS in response to ELR+CXC chemokines prior to the onset of EAE. Furthermore, IL-17, produced by infiltrating CD4+ T cells, can trigger upregulation of the ELR+CXC chemokines.

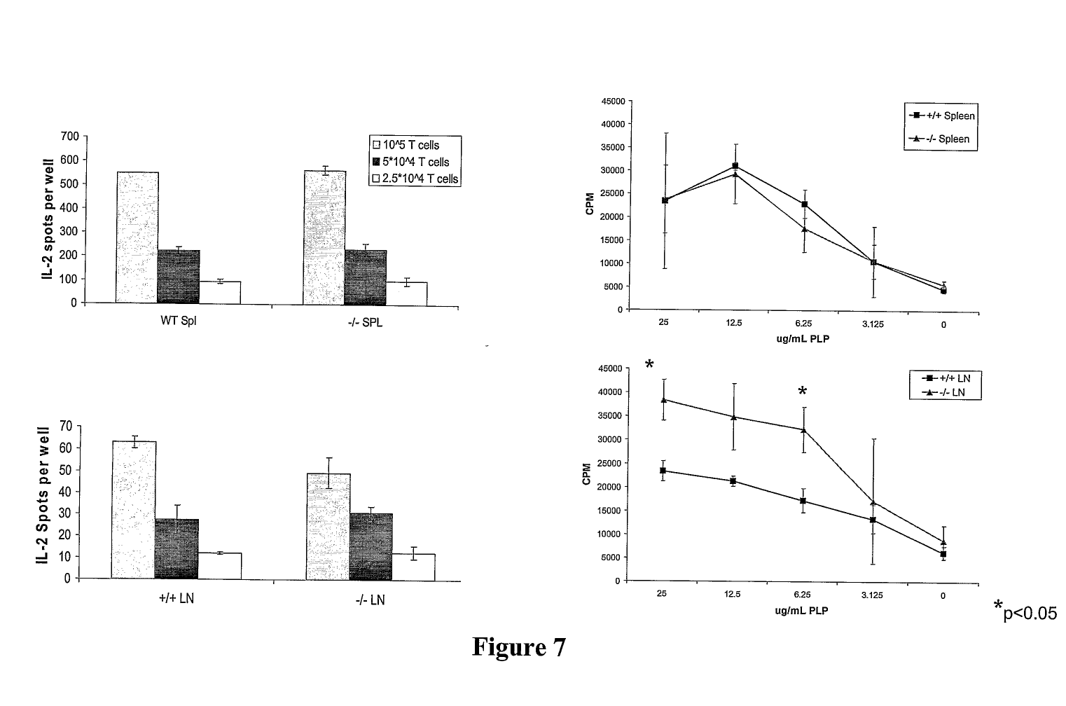

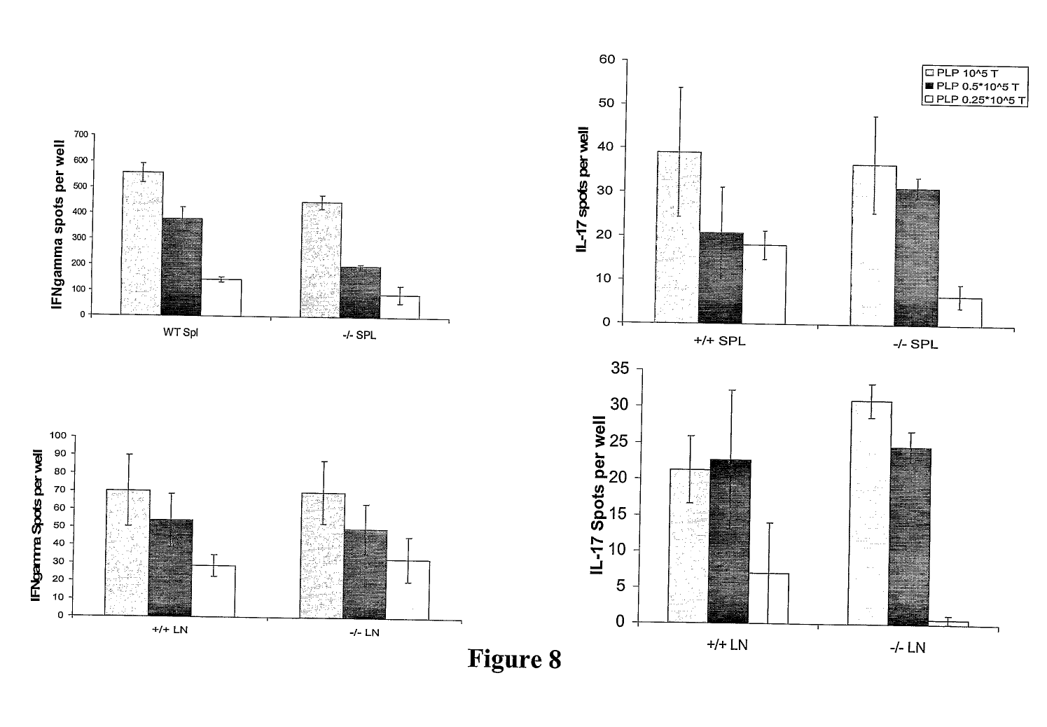

To determine whether peripheral T cells responses are diminished in CXCR2−/− mice, T-cells were harvested from the spleen and lymph nodes of wild type (WT) and CXC−/− mice and their proliferation was assessed following immunization with PLP₁₈₅₋₂₀₆ (FIG. 7). Results of the proliferation assay indicated that the rate of proliferation of PLP₁₈₅₋₂₀₆-specific T-cells in the spleen and lymph nodes was similar between WT and CXCR2 deficient mice. Additionally, inflammatory cytokines IL-2, IL-17 and IFN-γ were measured by ELISPOT assay (FIGS. 7 and 8). ELISPOT data showed that the presence or absence of CXCR2 did not affect the ability of T-cells to secrete cytokines as the levels of cytokine production for IL-2, IL-17 and IFN-γ was the same between WT and CXC2−/− mice. Hence, CXCR2−/− mice are fully capable of mounting peripheral immune responses against myelin antigens although they fail to form CNS infiltrates and are resistant to EAE induction. These results indicate that CXCR2 expression is important for the recruitment of neutrophils to the CNS during the preclinical phase of EAE, which is a critical step leading to the disruption of the blood-brain-barrier and subsequent development of perivascular lymphoid-myeloid inflammatory foci.

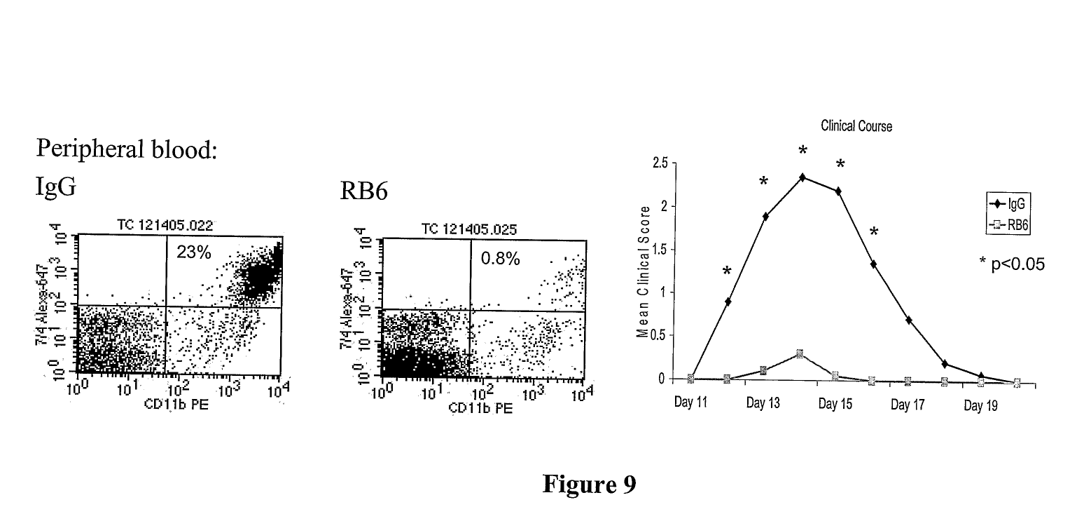

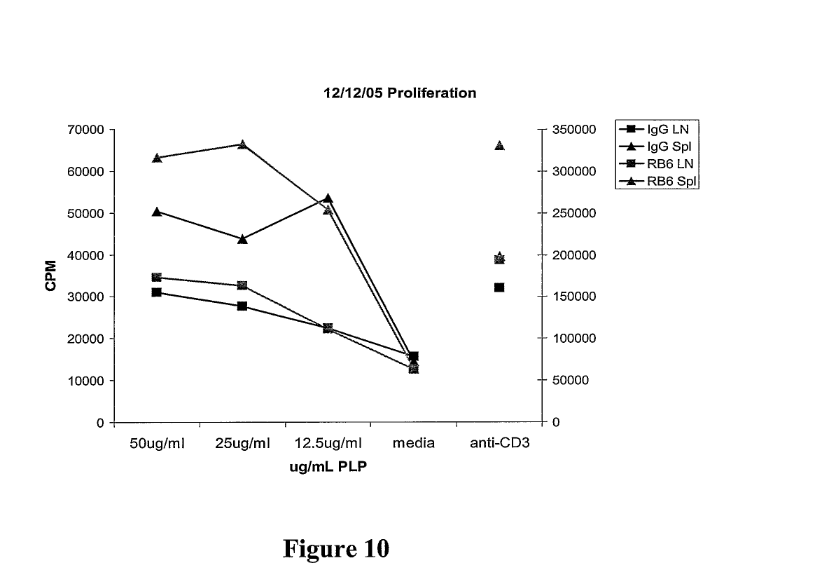

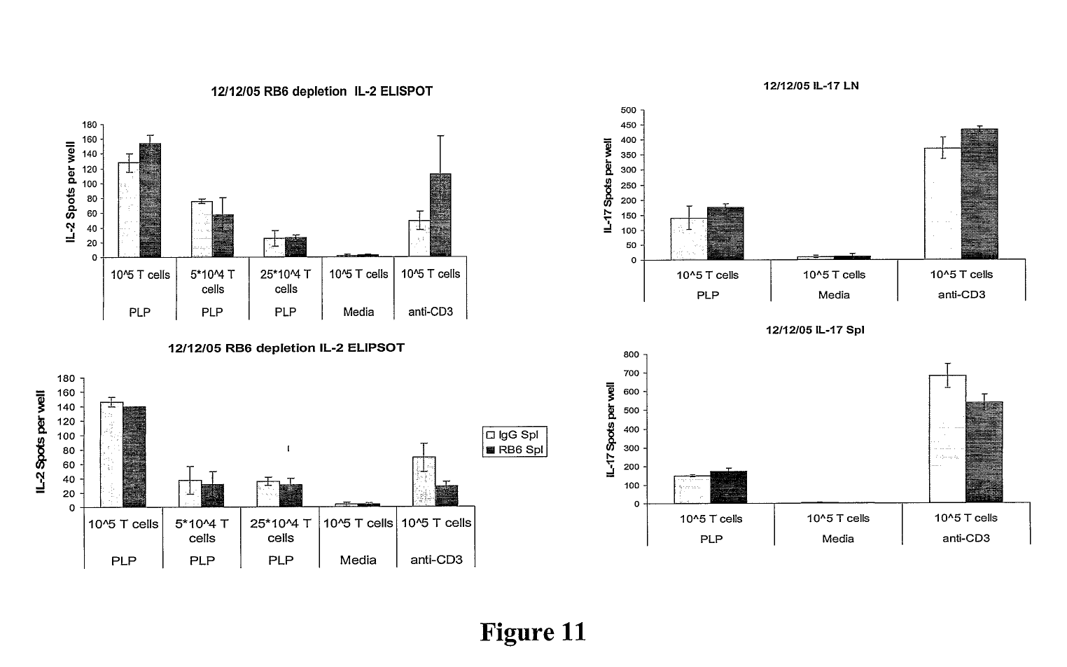

As the proliferation and cytokine secretion of T-cells did not vary between WT and CXCR2−/− mice, neutrophils were depleted in immunocompetent mice to investigate their effect on the clinical course of EAE. As seen in FIG. 9, RB6 (a monoclonal antibody that targets the neutrophil marker, Gr-1) was used to deplete neutrophils. SJL mice receiving RB6 were immunized with PLP₁₃₉₋₁₅₁ and compared with control mice receiving an irrelevant antibody. Mice receiving RB6 unlike control mice exhibited very mild or no signs of EAE. Additionally, T-cells were harvested from the spleen and lymph nodes of the mice from both groups and proliferation was assessed (FIG. 10). Neutrophil depletion had no effect on the proliferative ability of CD4+ T-cells. Furthermore, ELISPOTs measuring IL-2 and IL-17 production were performed showing no difference in IL-2 and IL-17 production between neutrophil depleted and control mice. These results indicate that neutrophils play a critical role in the establishment of CNS infiltrates and the development of clinical EAE. Furthermore, neutrophil depletion does not impair myelin peptide-immunized mice from mounting peripheral T cell responses. Hence, neutrophils can exert their functions during the effector phase of disease rather than during T cell priming.

### REFERENCES

Raine, C. S., Mokhtarian, F., and McFarlin, D. E. 1984. Adoptively transferred chronic relapsing experimental autoimmune encephalomyelitis in the mouse. Neuropathologic analysis. Laboratory Investigation 51:534-546.
Segal, B. M., and Shevach, E. M. 1996. IL-12 unmasks latent autoimmune disease in resistant mice. J Exp Med 184:771-775.
Segal, B. M., Chang, J. T., and Shevach, E. M. 2000. CpG oligonucleotides are potent adjuvants for the activation of autoreactive encephalitogenic T cells in vivo. J Immunol 164:5683-5688.
Segal, B. M., Dwyer, B., and Shevach, E. M. 1998. An IL-12/IL-10 Immunoregulatory Circuit Controls Susceptibility to Autoimmune Disease. J. Exp. Med. 187: 537.

1. A method of preventing an organ-specific autoimmune disease in a subject at risk for an organ-specific autoimmune disease comprising administering to the subject at risk for an organ-specific autoimmune disease an agent that blocks binding of an ELR+ (positive) CXC chemokine with a receptor.

2. The method of claim 1, wherein the autoimmune disease is selected from the group of autoimmune diseases consisting of rheumatoid arthritis, multiple sclerosis, acute disseminated encephalomyelitis, optic neuritis, transverse myelitis. Sjögren's syndrome, IBD, diabetes, thyroiditis, psoriasis, psoriatic arthritis, chronic inflammatory demyelinating polyneuropathy, systemic lupus erythematosis, sarcoidosis, Bechet's disease, vasculitides, and Guillain-Barre syndrome.

3. The method of claim 2, wherein the autoimmune disease is multiple sclerosis.

4. The method of claim 1, wherein the ELR+CXC chemokine is selected from the group of chemokines consisting of MIP-2, lipopolysaccharide-induced CXC chemokine (LIX), Interleukin-8 (IL-8), KC, neutrophil-activating protein-2 (NAP-2), growth-related oncogenes (GRO)-α, GRO-β, and GRO-γ, granulocyte chemotactic protein-2 (GCP-2), and epithelial neutrophil-activating protein 78 (ENA-78).

5. The method of claim 1, wherein the receptor is CXCR2 or CXCR1.

6. The method of claim 1, wherein the agent is an antibody.

7. A method of treating a subject with an organ-specific autoimmune disease comprising administering to a subject with an organ-specific autoimmune disease an agent that blocks the interaction of an ELR+ (positive) CXC chemokine with a receptor.

8. The method of claim 7, wherein the autoimmune disease is selected from the group of autoimmune diseases consisting of rheumatoid arthritis, acute disseminated encephalomyelitis optic neuritis, transverse myelitis, chronic inflammatory demyelinating polyneuropathy, Sjögren's syndrome, IBD, diabetes, thyroiditis, psoriasis, psoriatic arthritis and Guillain-Barre syndrome.

9. The method of claim 7, wherein the ELR+CXC chemokine is selected from the group of chemokines consisting of LIX, IL-8, MIP-2, KC, NAP-2, GRO-α, GRO-B, GRO-γ, GCP-2, and ENA-78.

10. The method of claim 7, wherein the receptor is CXCR2 or CXCR1.

11. The method of claim 7, wherein the agent is an antibody.

12. A method of diagnosing multiple sclerosis in a subject comprising detecting in the subject's cerebrospinal fluid an increase in the amount of ELR+CXC chemokines as compared to a control.

13. (canceled)

14. (canceled)

15. (canceled)

16. (canceled)

17. A method of preventing the infiltration of neutrophils into a tissue comprising inhibiting the interaction of ELR+CXC chemokines with a receptor by inhibiting production of the ELR+CXC chemokine.

18. The method of claim 17, wherein the ELR+CXC chemokine is selected from the group of chemokines consisting of MIP-2, LIX, IL-8, KC, NAP-2, GRO-α, GRO-B, GRO-γ, GCP-2, and ENA-78.

19. The method of claim 18, wherein the receptor is CXCR2 or CXCR1.

20. The method of claim 17, wherein the production of the ELR+CXC chemokine is inhibited by blocking IL-23 from binding IL-23R.

21. The method of claim 20, wherein IL-23 is blocked by binding IL-23 with an anti-IL-23 antibody.

22. The method of claim 17, wherein the production of the ELR+CXC chemokine is inhibited by blocking IL-17 from binding IL-17R.

23. The method of claim 22, wherein IL-17 is blocked by binding IL-17 with an anti-IL-17 antibody.

24. The method of claim 17, wherein the tissue is selected from the group consisting of nerve tissue, brain tissue, spinal cord tissue, lymphatic tissue, hepatic tissue, splenic tissue, pulmonary tissue, cardiac tissue, gastric tissue, intestinal tissue, pancreatic tissue, the thyroid gland, salivary glands, joints and the skin.

25. A method of screening for agents for treating an autoimmune disease comprising contacting a CXC receptor positive cell with the agent to be screened and detecting binding of ELR+CXC chemokines with the CXC receptor, wherein an agent that inhibits the interaction of the chemokine and the receptor can be used to treat the autoimmune disease.

26. The method of claim 25, wherein the CXC receptor is CXCR2 or CXCR1.

27. (canceled)

28. The method of claim 25, wherein the autoimmune disease is selected from the group of autoimmune diseases consisting of rheumatoid arthritis, multiple sclerosis, acute disseminated encephalomyelitis, optic neuritis, transverse myelitis, Sjögren's syndrome, IBD, diabetes, thyroiditis, psoriasis, psoriatic arthritis, chronic inflammatory demyelinating polyneuropathy, systemic lupus erythematosis, sarcoidosis, Bechet's disease, vasculitides, and Guillain-Barre syndrome.

29. The method of claim 28, wherein the autoimmune disease is multiple sclerosis.

30. (canceled)

31. (canceled)

32. (canceled)

33. (canceled)

34. (canceled)

35. The method of claim 25, wherein the CXC receptor positive cell is located in a subject with an autoimmune disease.

36. (canceled)

37. The method of claim 35, wherein the level of ELR+CXC chemokines are monitored in the affected tissue of the subject.

38. The method of claim 37, wherein the tissue is selected from the group consisting of nerve tissue, brain tissue, spinal cord tissue, lymphatic tissue, hepatic tissue, splenic tissue, pulmonary tissue, cardiac tissue, gastric tissue, intestinal tissue, pancreatic tissue, the thyroid gland, salivary glands, joints and the skin.

39. The method of claim 35, wherein the subject is a mammal.

40. The method of claim 39, wherein the subject is a mouse.

41. (canceled)

42. The method of claim 39, wherein the subject is a human.

43. The method of claim 35, wherein the agent is an antibody.
